(19) United States
(12) Patent Application Publication
(10) Pub. No.: US 2021/0386351 A1
(43) Pub. Date: Dec. 16, 2021

(54) BRAIN-COMPUTER INTERFACE APPARATUS FOR MINIMIZING SIGNAL CORRECTION PROCESS BETWEEN USERS USING CLUSTERING TECHNOLOGY BASED ON BRAIN ACTIVITY AND METHOD OF OPERATING THE SAME

(71) Applicant: Korea University Research and Business Foundation, Seoul (KR)

(72) Inventors: Dong Joo KIM, Seoul (KR); Se Ho LEE, Seoul (KR); Young Tak KIM, Nonsan-si (KR)

(73) Assignee: Korea University Research and Business Foundation, Seoul (KR)

(21) Appl. No.: 17/155,281

(22) Filed: Jan. 22, 2021

(30) Foreign Application Priority Data
Jun. 12, 2020 (KR) 10-2020-0071772
Oct. 29, 2020 (KR) 10-2020-0142178

(51) Int. Cl. A61B 5/31; A61B 5/291; A61B 5/00; G06K 9/62

(52) U.S. Cl. CPC A61B 5/31; A61B 5/291; G06K 9/6223; A61B 5/7475; A61B 5/7203

(57) ABSTRACT

The present disclosure relates to a technical idea for minimizing a signal correction process between users using brain activity-based clustering technology. More specifically, the present disclosure relates to technology for minimizing a signal correction process between users by clustering a brain signal of a measurement subject into a specific clustering model and determining an intention of the measurement subject using an intention determination model learned on the specific clustering model. The brain-computer interface apparatus according to one embodiment of the present disclosure may include a feature extractor for extracting a plurality of clustering features using frequency powers for each band of brain signals measured from a plurality of learning subjects; a clustering model generator for generating a plurality of clustering models based on the extracted clustering features; and a brain wave processor for constructing an intention determination model by performing machine learning of brain signals for each of the generated clustering models, determining a newly measured brain signal of a measurement subject as any one of the clustering models, and determining an intention of the measurement subject using the constructed intention determination model.

### CROSS-REFERENCE TO RELATED APPLICATIONS

This application claims priority to Korean Patent Application No. 10-2020-0071772, filed on Jun. 12, 2020, and Korean Patent Application No. 10-2020-0142178, filed on Oct. 29, 2020, in the Korean Intellectual Property Office, the disclosures of each of which are incorporated herein by reference.

### BACKGROUND OF THE DISCLOSURE

### Field of the Disclosure

The present disclosure relates to a technical idea for minimizing a signal correction process between users using clustering technology based on brain activity. More specifically, the present disclosure relates to technology for minimizing a signal correction process between users by clustering a brain signal of a measurement subject into a specific clustering model and determining an intention of the measurement subject using an intention determination model learned on the specific clustering model.

### Description of the Related Art

Brain-computer interfaces (BCIs), which have been actively developed in recent years, include an interface that uses change in brain waves caused by external stimuli and an interface that uses change in brain waves caused by user's intrinsic change and motor imagery.

The brain-computer interface technology is a new communication technology that aims to directly connect the brain and a computer.

The brain-computer interface technology may use technology for measuring electroencephalogram (EEG) signals.

An EEG signal refers to a signal obtained by measuring electrical phenomena that appear in the cortex of the brain according to brain activity.

Methods of measuring EEG signals include invasive and non-invasive methods.

As a representative non-invasive method, there is a method of measuring EEG signals by measuring brain activity by placing electrodes on the scalp.

The non-invasive method has a disadvantage in that EEG signals having a low signal-to-noise ratio (SNR) are obtained, but has advantages of high resolution in a temporal domain and low cost.

EEG signals are susceptible to contamination by eye blinking, movement, or external noise. In addition, since the magnitude of measured EEG signals is small, the brain-computer interface technology using brain signals still has difficulties.

For example, the data of EEG signals measured from subject may be changed by eye blinking or noise from the outside.

Accordingly, it may be difficult to provide highly reliable brain-computer interface technology using EEG signals.

To develop brain-computer interface technology based on motor imagery, a temporal domain, a spectral domain, and a spatial domain are separated, and research on each domain is actively conducted.

With the recent development of deep learning (DL) technology, the accuracy of brain wave analysis is improved. However, due to the characteristic of deep learning, studies using only specific domains are being conducted.

Accordingly, it is necessary to improve performance by fusing the features of various domains. In addition, it is necessary to design a new deep learning model specialized in various domains.

When measuring EEG signals, even if people think the same thoughts, the electrical characteristics of the brain cortex differ among subjects. This is called “inter-subject variability (ISV)”.

Due to the diversity of the types of brain waves measured EEG signals, when applying brain-computer interface technology, a process of sufficiently measuring user's brain waves and creating a customized model for each user is required. However, this process takes a lot of time.

### RELATED ART DOCUMENTS

### Patent Documents

Korean Patent No. 10-2118757 “CORRELATION OF BRAIN SIGNALS TO INTENTIONAL AND UNINTENTIONAL CHANGES IN BRAIN CONDITIONS”

Korean Patent Application Publication No. 10-2020-0099811 “DEVICE AND METHOD FOR PROVIDING DIAGNOSIS INFORMATION FOR EPILEPSY USING RESTING BRAIN WAVES”

Korean Patent No. 10-1675875 “METHOD AND SYSTEM FOR RETRIEVING ELECTROENCEPHALOGRAM SIGNALS USING SPECTRUM ANALYSIS AND VECTOR QUANTIZATION”

US Patent Application Publication No. 2020/0023189 “BRAIN COMPUTER INTERFACE SYSTEMS AND METHODS OF USE THEREOF”

### SUMMARY OF THE DISCLOSURE

Therefore, the present disclosure has been made in view of the above problems, and it is an object of the present disclosure to provide a brain-computer interface apparatus capable of minimizing a signal correction process between users using clustering technology based on brain activity and a method of operating the same.

It is another object of the present disclosure to provide a brain-computer interface apparatus capable of minimizing, without deterioration in performance, a signal correction process by using clustering models designed using the brain signals of a user rather than individual customized models that take a lot of time and a method of operating the brain-computer interface apparatus.

It is still another that object of the present disclosure to provide a brain-computer interface apparatus have high recognition performance for user intentions, while a signal correction process was minimized by using clustering models and a method of operating the brain-computer interface apparatus.

It is yet another object of the present disclosure to reduce time spent on classifying user intentions by extracting features for clustering models using the frequency powers of brain signals measured from a plurality of learning subjects or a measurement subject.

In accordance with one aspect of the present disclosure, a brain-computer interface apparatus is providing capable of minimizing a signal correction process between users using clustering technology based on brain activity, the brain-computer interface apparatus including a feature extractor for extracting a plurality of clustering features using frequency powers for each band of brain signals measured from a plurality of subjects; a clustering model generator for generating a plurality of clustering models based on the extracted clustering features; and a brain wave processor for constructing an intention determination model by performing machine learning of brain signals for each of the generated clustering models, determining a newly measured brain signal of a measurement subject as any one of the clustering models, and determining an intention of the measurement subject using the constructed intention determination model.

According to one embodiment of the present disclosure, the brain-computer interface apparatus may further include a brain wave measurement device for, using a plurality of measurement electrodes attached to a plurality of regions of the learning subjects or the measurement subject, measuring a plurality of brain signals for each of the regions.

The brain signals may exhibit different frequency powers according to the regions. The brain wave measurement device may measure brain signals having frequency powers of a first band at which a frequency power of 8 Hz to 12 Hz is measured, a second band at which a frequency power of 12 Hz to 18 Hz is measured, and a third band at which a frequency power of 18 Hz to 30 Hz is measured according to positions of the regions.

The feature extractor may extract at least one clustering feature of first clustering features corresponding to the first band, second clustering features corresponding to the second band, and third clustering features corresponding to the third band.

The feature extractor may determine a plurality of clustering features for each band by averaging power spectral density (PSD) of the frequency powers for each band, and may determine two clustering features among the determined clustering features through principal component analysis (PCA).

The clustering model generator may generate the clustering models using the determined two clustering features and cluster analysis (k-means clustering).

The brain wave processor may determine an intention of the measurement subject using an intention determination model corresponding to the determined clustering model, and thus may reduce the signal correction process by reducing a machine learning process for the newly measured brain signal of the measurement subject.

The clustering model generator may generate the clustering models so that inter-subject variability (ISV) of each of the clustering models is determined to be smaller than inter-subject variability (ISV) of all of the learning subjects.

In accordance with another aspect of the present disclosure, provided is a method of operating a brain-computer interface apparatus capable of minimizing a signal correction process between users using clustering technology based on brain activity, the method including extracting, a plurality of clustering features using frequency powers for each band of brain signals measured from a plurality of subjects; generating, in a clustering model generator, a plurality of clustering models based on the extracted clustering features; and constructing an intention determination model by performing machine learning of brain signals for each of the generated clustering models, determining a newly measured brain signal of a measurement subject as any one of the clustering models, and determining an intention of the measurement subject using the constructed intention determination model, in a brain wave processor.

According to one embodiment of the present disclosure, the method further includes measuring, in a brain wave measurement device, a plurality of brain signals for each of a plurality of regions of the learning subjects or the measurement subject by using a plurality of measurement electrodes attached to the regions.

The brain signals may exhibit different frequency powers according to the regions. The measuring of a plurality of brain signals for each of the regions may include measuring brain signals having frequency powers of a first band at which a frequency power of 8 Hz to 12 Hz is measured, a second band at which a frequency power of 12 Hz to 18 Hz is measured, and a third band at which a frequency power of 18 Hz to 30 Hz is measured according to positions of the regions. The determining of an intention of the measurement subject using the constructed intention determination model may include reducing the signal correction process by reducing a machine learning process for the newly measured brain signal of the measurement subject as an intention of the measurement subject is determined using an intention determination model corresponding to the determined clustering model.

### BRIEF DESCRIPTION OF THE DRAWINGS

The above and other objects, features and other advantages of the present disclosure will be more clearly understood from the following detailed description taken in conjunction with the accompanying drawings, in which:

FIG. 1 is a diagram for explaining a brain-computer interface apparatus according to one embodiment of the present disclosure;

FIGS. 2A and 2B are diagrams for explaining an example in which a plurality of clustering features is extracted by a brain-computer interface apparatus according to one embodiment of the present disclosure;

FIGS. 3A to 3C are diagrams for explaining examples in which a plurality of clustering models is generated by a brain-computer interface apparatus according to one embodiment of the present disclosure;

FIGS. 4A and 4B are diagrams for comparing inter-subject variability (ISV) for each frequency band according to a conventional technology and inter-subject variability (ISV) for each frequency band based on clustering models according to one embodiment of the present disclosure;

FIG. 5 is a diagram for comparing the classification accuracy of a brain-computer interface apparatus according to one embodiment of the present disclosure and the classification accuracy of a conventional technology; and

FIG. 6 is a flowchart for explaining a method of operating a brain-computer interface apparatus according to one embodiment of the present disclosure.

### DETAILED DESCRIPTION OF THE DISCLOSURE

Hereinafter, the embodiments of the present disclosure will be described in detail with reference to the drawings.

It should be understood that the present disclosure is not limited to the embodiments according to the concept of the present disclosure, but includes changes, equivalents, or alternatives falling within the spirit and scope of the present disclosure.

In the following description of the present disclosure, detailed description of known functions and configurations incorporated herein will be omitted when it may make the subject matter of the present disclosure unclear.

In addition, the terms used in the specification are defined in consideration of functions used in the present disclosure, and can be changed according to the intent or conventionally used methods of clients, operators, and users. Accordingly, definitions of the terms should be understood on the basis of the entire description of the present specification.

In description of the drawings, like reference numerals may be used for similar elements. The singular expressions in the present specification may encompass plural expressions unless clearly specified otherwise in context.

In this specification, expressions such as “A or B” and “at least one of A and/or B” may include all possible combinations of the items listed together.

Expressions such as “first” and “second” may be used to qualify the elements irrespective of order or importance, and are used to distinguish one element from another and do not limit the elements.

It will be understood that when an element (e.g., first) is referred to as being “connected to” or “coupled to” another element (e.g., second), it may be directly connected or coupled to the other element or an intervening element (e.g., third) may be present.

As used herein, “configured to” may be used interchangeably with, for example, “suitable for”, “ability to”, “changed to”, “made to”, “capable of”, or “designed to” in terms of hardware or software.

In some situations, the expression “device configured to” may mean that the device “may do ˜” with other devices or components.

For example, in the sentence “processor configured to perform A, B, and C”, the processor may refer to a general purpose processor (e.g., CPU or application processor) capable of performing corresponding operation by running a dedicated processor (e.g., embedded processor) for performing the corresponding operation, or one or more software programs stored in a memory device.

In addition, the expression “or” means “inclusive or” rather than “exclusive or”. That is, unless otherwise mentioned or clearly inferred from context, the expression “x uses a or b” means any one of natural inclusive permutations.

Terms, such as “unit” or “module”, etc., should be understood as a unit that processes at least one function or operation and that may be embodied in a hardware manner, a software manner, or a combination of the hardware manner and the software manner.

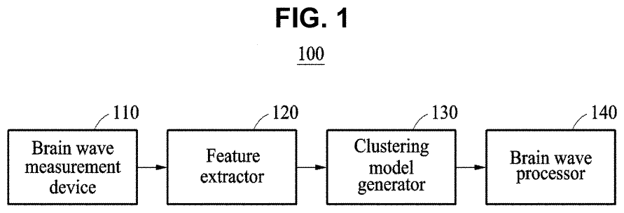

FIG. 1 is a diagram for explaining a brain-computer interface apparatus according to one embodiment of the present disclosure.

FIG. 1 illustrates the components of a brain-computer interface apparatus according to one embodiment of the present disclosure.

Referring to FIG. 1, a brain-computer interface apparatus 100 according to one embodiment of the present disclosure may be a brain-computer interface apparatus capable of minimizing a signal correction process between users using brain activity-based clustering technology.

According to one embodiment of the present disclosure, the brain-computer interface apparatus 100 includes a brain wave measurement device 110, a feature extractor 120, a clustering model generator 130, and a brain wave processor 140.

For example, using a plurality of measurement electrodes attached to a plurality of regions of a plurality of learning subjects or a measurement subject, the brain wave measurement device 110 may measure a plurality of brain signals for each region.

For example, a plurality of brain signals may exhibit different frequency powers according to a plurality of regions.

In addition, the brain signals may include electroencephalogram (EEG) signals. According to one embodiment of the present disclosure, the brain wave measurement device 110 may measure brain signals having the frequency powers of a first band at which a frequency power of 8 Hz to 12 Hz is measured, a second band at which a frequency power of 12 Hz to 18 Hz is measured, and a third band at which a frequency power of 18 Hz to 30 Hz is measured according to the positions of a plurality of regions.

For example, the first band may be referred to as an a band, the second band may be referred to as a low β band, and the third band may be referred to as a high 13 band.

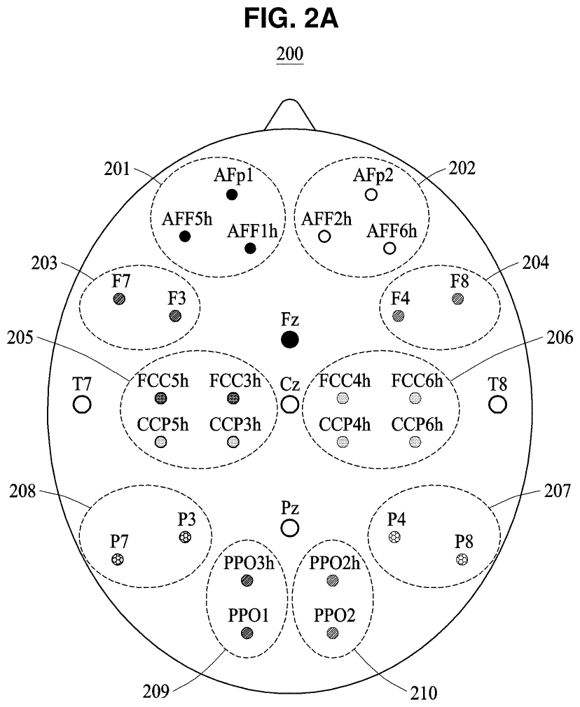

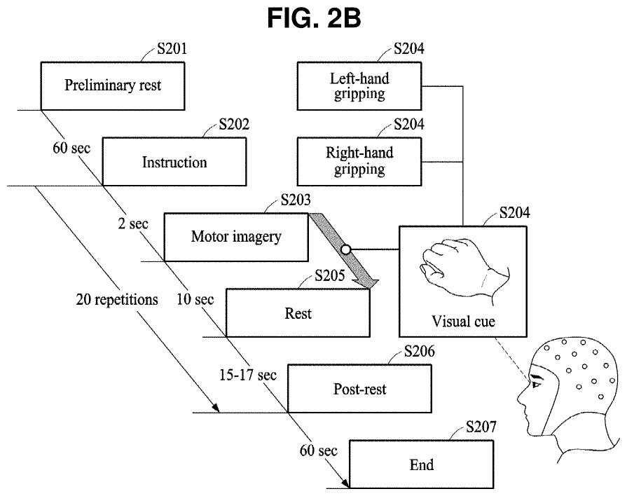

For example, the frequency powers of the first to third bands may represent results calculated at different positions, and frequency powers according to different positions will be additionally described with reference to FIG. 2A.

For example, the standard deviation of power distribution in the first to third bands may be used to evaluate inter-subject variability (ISV).

According to one embodiment of the present disclosure, the feature extractor 120 may extract a plurality of clustering features using frequency powers for each band of brain signals measured from a plurality of learning subjects.

For example, the feature extractor 120 may extract at least one clustering feature of a first clustering feature corresponding to the first band, a second clustering feature corresponding to the second band, and a third clustering feature corresponding to the third band.

That is, the feature extractor 120 may extract a clustering feature for each band. For example, the clustering features may be used as data for generating a clustering model. According to one embodiment of the present disclosure, the feature extractor 120 may determine a plurality of clustering features for each band by averaging the power spectral density (PSD) of the frequency powers for each band.

That is, the feature extractor 120 may determine a plurality of clustering features according to the frequency power of each band.

According to one embodiment of the present disclosure, the feature extractor 120 may determine two clustering features among a plurality of clustering features determined through principal component analysis (PCA).

For example, the clustering model generator 130 may generate a plurality of clustering models based on a plurality of clustering features extracted by the feature extractor 120.

That is, the clustering model generator 130 may generate a plurality of clustering models using two clustering features determined by the feature extractor 120 and cluster analysis (k-means clustering).

According to one embodiment of the present disclosure, the clustering model generator 130 may generate a plurality of clustering models so that the inter-subject variability (ISV) of each of a plurality of clustering models is determined to be smaller than the inter-subject variability (ISV) of all of a plurality of learning subjects.

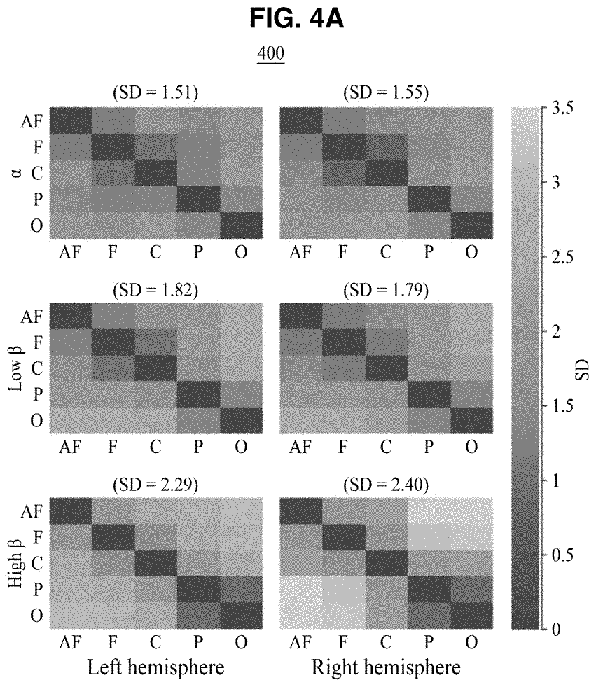

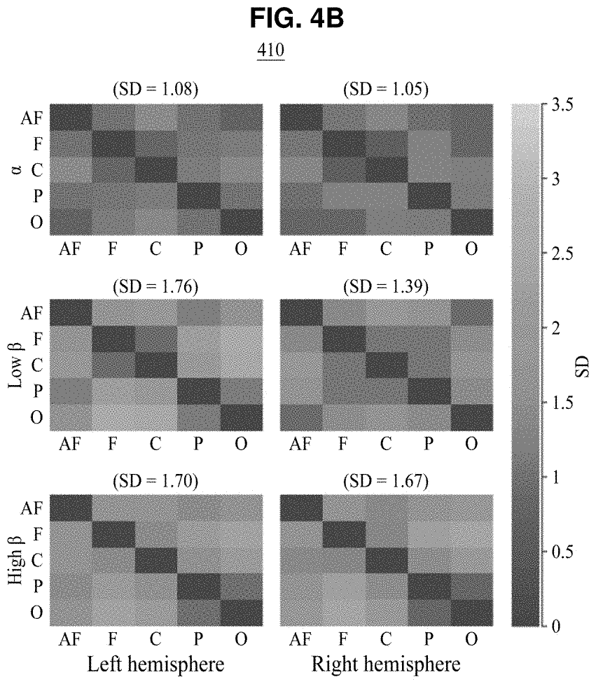

For example, comparison results between the inter-subject variability (ISV) of each of a plurality of clustering models and the inter-subject variability (ISV) of all of a plurality of learning subjects will be additionally described with reference to FIGS. 4A and 4B.

According to one embodiment of the present disclosure, the brain wave processor 140 may construct an intention determination model by performing machine learning of brain signals for each of a plurality of clustering models generated by the clustering model generator 130, determine a newly measured brain signal of a measurement subject as any one of the clustering models, and determine an intention of the measurement subject using the constructed intention determination model.

For example, the brain wave processor 140 may extract features for constructing an intention determination model using the common spatial pattern (CSP) of brain signals for each of a plurality of clustering models.

According to one embodiment of the present disclosure, the brain wave processor 140 may extract the analysis results of a plurality of brain waves by applying the brain wave signals for each of a plurality of clustering models to a common spatial pattern (CSP) filter, generate matrix data in descending order from differences in the extracted analysis results of the brain waves, and extract features spatially classified from each other for the generated matrix data as features for constructing an intention determination model.

The brain-computer interface apparatus 100 according to one embodiment of the present disclosure constructs a subject using the frequency pattern of a brain signal and uses a common spatial pattern to learn an intention determination model.

According to one embodiment of the present disclosure, assuming that T samples are measured from N electrodes, the brain wave processor 140 may generate a matrix of N×T when one measurement is performed.

In addition, the brain wave processor 140 may calculate a covariance of a matrix of N×T that appears when X tasks are performed when NN tasks are performed.

For example, when one task is performed, a covariance may be calculated by Equation 1 below.

$$C = \frac{{S}^{′}}{trans ( {S}^{′} )} \qquad [ Equation 1 ]$$

In Equation 1, C may represent a covariance, and S′ may represent a matrix of N×T.

In this way, when the covariance (C) is calculated, N×N matrices are derived, and the number of measurements related to the covariance (C) may be expressed as n.

Since the brain wave processor 140 has performed X tasks, the brain wave processor 140 may repeatedly calculate the average of a covariance (C1) for an X₁th task and the average of a covariance (C2) for an X₂th task to calculate a covariance (Ci) for an Xᵢth task.

The brain-computer interface apparatus may generate a whitening transformation matrix (Q) by adding all the averages of the obtained covariances and converting the sum (Csum) of the averages of the covariances using Equation 2 below.

Q=\sqrt{λ⁻¹}U′ [Equation 2]

In Equation 2, Q may represent a whitening transformation matrix, U may represent an eigenvector, and λ may represent an eigenvalue.

The brain wave processor 140 causes the whitening transformation matrix Q calculated using Equation 2 to have the same eigenvectors for each class mean covariance matrix.

Accordingly, to have different covariances by applying this whitening matrix, Equation 3 must be derived.

Sᵢ=QC̅ₗQ′ [Equation 3]

In Equation 3, S may represent a matrix of N×T, Q may represent a covariance, and Ci may represent the sum of the averages of covariances.

Here, when there are two classes, Equation 4 may be derived.

S1=Bλ₁B′,S2=Bλ₂B′ [Equation 4]

In Equation 4, S1 may represent the matrix of N×T of a first class, B may represent an eigenvector transformation variable, and S2 may represent the matrix of N×T of a second class.

The brain wave processor 140 may obtain a whitening transformation matrix (Q) equal to the weight (W) of a CSP filter obtained by using S1 and S2 obtained in this way, and may calculate a feature point (Z) that has passed through the CSP filter by multiplying the weight (W) and the signal of an original signal N×T.

According to one embodiment of the present disclosure, the brain wave processor 140 may extract N×T results for a signal that has passed through the CSP filter, and after making the one with the largest difference between the two in order based on the extracted results, the extracted features are listed in one block of 8×8.

Next, the brain wave processor 140 may extract the features of the brain waves of a discrete moment by extracting features spatially classified from each other for a plurality of tasks, and may classify the intentions of a measurement subject by applying the extracted features to RBF-SVM.

For example, the brain wave processor 140 may distinguish two classes of data by variance and maximize a difference between the two classes through a CSP filter.

According to one embodiment of the present disclosure, the brain wave processor 140 may construct an intention determination model by performing machine learning of features extracted through the CSP filter using any one support vector machine of a linear support vector machine (SVM), a poly support vector machine (SVM), a radial basis function (RBF) support vector machine (SVM), and a sigmoid support vector machine (SVM).

For example, the brain wave processor 140 may determine the intention of a measurement subject using an intention determination model corresponding to a clustering model, and thus may reduce a machine learning process for a newly measured brain signal of the measurement subject to reduce a signal correction process.

Accordingly, the present disclosure may provide a brain-computer interface apparatus capable of minimizing a signal correction process between users using brain activity-based clustering technology.

FIGS. 2A and 2B are diagrams for explaining an example in which a plurality of clustering features are extracted by a brain-computer interface apparatus according to one embodiment of the present disclosure.

FIG. 2A illustrates brain regions subdivided when the brain-computer interface apparatus according to one embodiment of the present disclosure measures a brain signal using a plurality of measurement electrodes.

Referring to FIG. 2A, a brain region 200 may be divided into a first region 201, a second region 202, a third region 203, a fourth region 204, a fifth region 205, a sixth region 206, a seventh region 207, an eighth region 208, a ninth region 209, and a tenth region 210.

For example, the first region 201 may correspond to a left anterior frontal region, the second region 202 may correspond to a right anterior frontal region, the third region 203 may correspond to a left frontal region, and the fourth region 204 may correspond to a right frontal region.

In addition, the fifth region 205 may correspond to a left central region, the sixth region 206 may correspond to a right central region, the seventh region 207 may correspond to a right parietal region, the eighth region 208 may correspond to a left parietal region, the ninth region 209 may correspond to a left occipital region, and the tenth region 210 may correspond to a right occipital region.

The brain-computer interface apparatus according to one embodiment of the present disclosure may generate a plurality of clustering models using principal component analysis (PCA) based on power spectral density (PSD) calculated in each region, and may perform clustering according to the generated models.

For example, the brain-computer interface apparatus performs grouping into 10 regions according to the positions of electrodes.

For example, the 10 groups may be composed of 2 to 4 electrodes.

The brain-computer interface apparatus may derive 10 features for each frequency band by averaging the power spectral densities of brain signals measured through a plurality of electrodes in each of 10 regions. That is, brain-computer interface apparatus may derive a total of 30 features.

The brain-computer interface apparatus according to one embodiment of the present disclosure may calculate two main features PC1 and PC2 among 10 features by performing principal component analysis (PCA) on 10 features for each band, and may generate three clustering models using two features and cluster analysis (k-means clustering).

Here, the three clustering models are clustered as brain signals having a small difference in power spectral density for each clustering model.

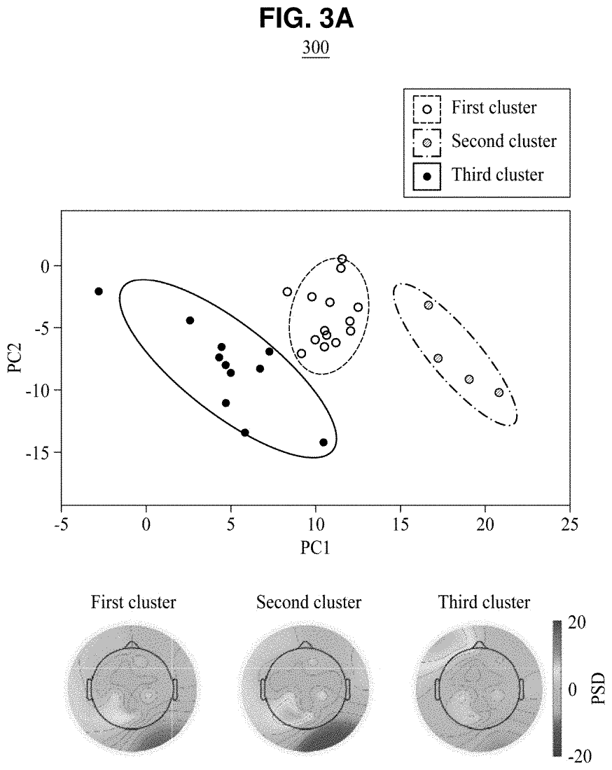

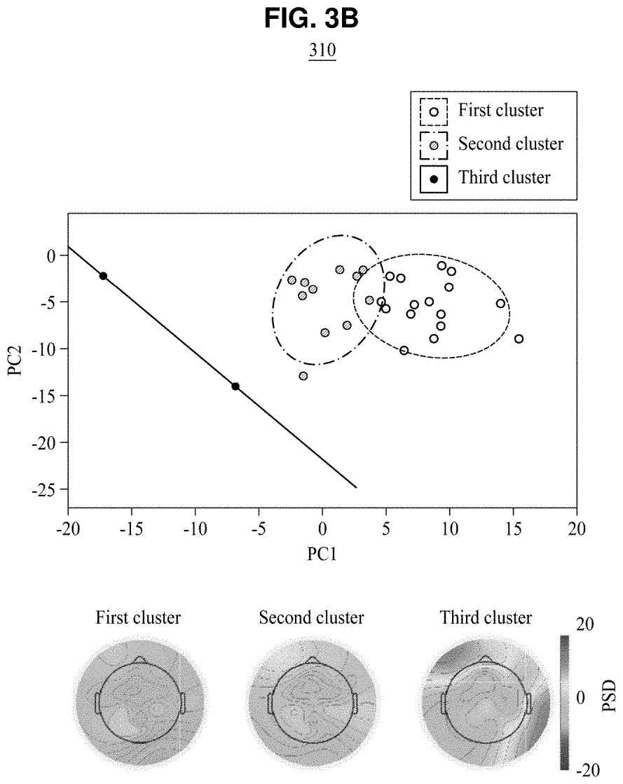

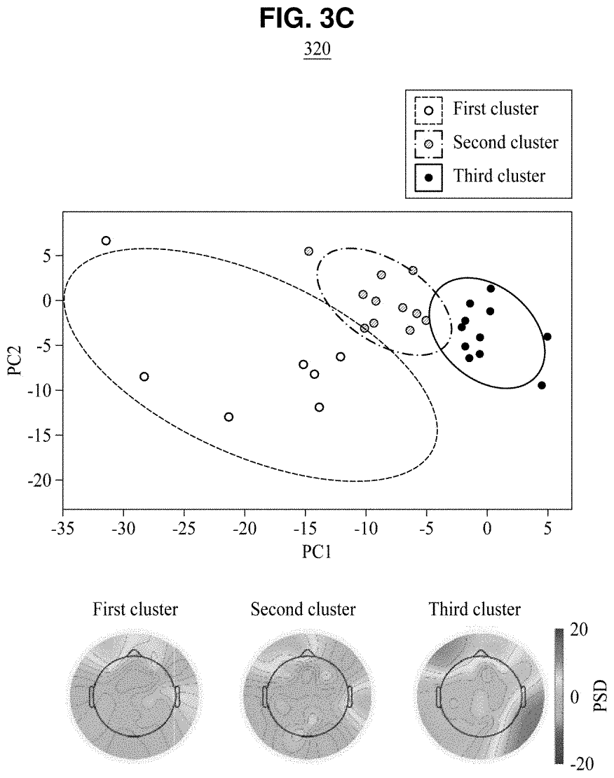

Clustering results according to the results of generating clustering models will be additionally described with reference to FIGS. 3A to 3C.

FIG. 2B illustrates a process in which the brain-computer interface apparatus according to one embodiment of the present disclosure measures brain signals from a plurality of learning subjects or a measurement subject to extract a plurality of clustering features.

Referring to FIG. 2B, a learning subject takes a preliminary rest in step S201. After 60 seconds have elapsed, the learning subject receives an instruction in step S202. After 2 seconds have elapsed, the learning subject performs motor imagery (MI) in step S203. After 10 seconds have elapsed, the learning subject takes a rest in step S205.

Between step S203 and step S205, the learning subject imagines a left-hand gripping or right-hand gripping motion corresponding to step S204, and performs motor imagery related to a visual cue.

After 15 seconds to 17 seconds have elapsed, the learning subject takes a post-rest in step S206, and after 60 seconds have elapsed, the brain signal measurement procedure ends in step 207.

Here, brain wave measurement electrodes may be positioned on the head of the learning subject, and may measure a plurality of brain signals from a plurality of measurement points. The brain wave measurement electrodes may record a data set at a sampling rate of 1,000 Hz, perform downsampling at 200 Hz, and then acquire 2,000 samples per test.

For example, the brain-computer interface apparatus may acquire brain signals having relatively high quality when the learning subject is in a resting state.

Here, artifacts may be removed from the acquired brain signals, and brain signals in a temporal domain may be transformed into a spectral domain by fast Fourier transform (FFT).

FIGS. 3A to 3C are diagrams for explaining examples in which a plurality of clustering models is generated by a brain-computer interface apparatus according to one embodiment of the present disclosure.

Referring to FIGS. 3A to 3C, power distribution for each band may exhibit various patterns in clusters and frequency bands.

FIG. 3A is a graph 300 showing analysis results in relation to the main clustering features PC1 and PC2 of principal component analysis (PCA) in first to third clustering models acquired in a first band region by the brain-computer interface apparatus according to one embodiment of the present disclosure.

Referring to the graph 300 of FIG. 3A, in the case of a first band, the power distribution of the left frontal lobe and the parietal occipital region differs between the first cluster and the second cluster.

In addition, the third cluster showed high activation in relation to power spectral density (PSD) in the left front.

FIG. 3B is a graph 310 showing analysis results in relation to the main clustering features PC1 and PC2 of principal component analysis (PCA) in first to third clustering models acquired in a second band region by the brain-computer interface apparatus according to one embodiment of the present disclosure.

Referring to the graph 310 of FIG. 3B, in the case of a second band, the distribution activation pattern of power spectral density (PSD) across the entire right occipital region in the third cluster clearly shows a difference.

FIG. 3C is a graph 320 showing analysis results in relation to the main clustering features PC1 and PC2 of principal component analysis (PCA) in first to third clustering models acquired in a third band region by the brain-computer interface apparatus according to one embodiment of the present disclosure.

Referring to the graph 320 of FIG. 3C, in the case of a third band, the distribution of power spectral density (PSD) in the left and right temporal regions shows high activation.

FIGS. 4A and 4B are diagrams for comparing inter-subject variability (ISV) for each frequency band according to a conventional technology and inter-subject variability (ISV) for each frequency band based on clustering models according to one embodiment of the present disclosure.

FIG. 4A shows the standard deviation of the frequency power difference between brain regions in a first band, a second band, and a third band for total subjects without clustering, and FIG. 4B shows the standard deviation of the frequency power difference between brain regions in a first band, a second band, and a third band for clustered subjects.

Referring to a graph 400 of FIG. 4A, the first band may correspond to an a band, the second band may correspond to a low β band, and the third band may correspond to a high β band.

In addition, in the graph 400, SD may represent standard deviation, AF may represent an anterior frontal brain region, F may represent a frontal brain region, C may represent a central brain region, P may represent a parietal brain region, and O may represent the brain region of a larynx region.

Referring to the graph 400, in the first band, the standard deviations of the left and right hemispheres may be 1.51 and 1.55, respectively. In the second band, the standard deviations of the left and right hemispheres may be 1.82 and 1.79, respectively. In the third band, the standard deviations of the left and right hemispheres may be 2.29 and 2.40, respectively.

Referring to a graph 410 of FIG. 4B, the first band may correspond to an a band, the second band may correspond to a low β band, and the third band may correspond to a high β band.

In addition, in the graph 410, SD may represent standard deviation, AF may represent an anterior frontal brain region, F may represent a frontal brain region, C may represent a central brain region, P may represent a parietal brain region, and O may represent the brain region of a larynx region.

Referring to the graph 410, in the first band, the standard deviations of the left and right hemispheres may be 1.08 and 1.05, respectively. In the second band, the standard deviations of the left and right hemispheres may be 1.76 and 1.39, respectively. In the third band, the standard deviations of the left and right hemispheres may be 1.70 and 1.67, respectively.

When the standard deviations of the graph 400 and the graph 410 are compared, the clustered subjects exhibit a relatively low standard deviation.

In particular, the standard deviations of the left and right hemispheres in the first band of the graph 410 are 1.08 and 1.05, respectively, exhibiting the lowest standard deviations.

That is, the inter-subject variability (ISV) of clustered subjects using clustering models may smaller than the inter-subject variability (ISV) without cluster division for total subjects.

Accordingly, the present disclosure may provide a brain-computer interface apparatus having high recognition performance for user intentions while minimizing a signal correction process by using customized clustering models and a method of operating the brain-computer interface apparatus.

In addition, the present disclosure may reduce time spent on classifying user intentions by extracting clustering features for designing clustering models using the frequency powers of brain signals measured from a plurality of learning subjects or a measurement subject.

In addition, in the case of a conventional technology, since a process of separately learning a model suitable for a subject is required, prior learning is required to learn a model suitable for a corresponding subject.

However, in the case of the present disclosure, brain waves may be analyzed in the resting state of a subject, and it may be determined in which clustering model the brain signals of the subject are included. When the clustering model is determined, the intention of the subject may be determined using an intention determination model learned in advance using subjects belonging to a corresponding group.

Accordingly, in the case of the present disclosure, even when a subject is measured for the first time, the intention of the subject may be determined without repetitive brain wave measurement to learn an intention determination model for determining an intention. Accordingly, the time for learning a model suitable for a subject may be reduced.

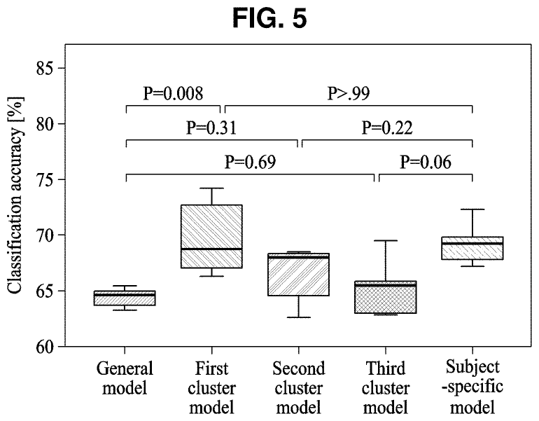

FIG. 5 is a diagram for comparing the classification accuracy of a brain-computer interface apparatus according to one embodiment of the present disclosure and the classification accuracy of a conventional technology.

Referring to FIG. 5, in a graph 500, the vertical axis represents classification accuracy, and the horizontal axis shows a general model, a first cluster model, a second cluster model, a third cluster model, and a subject-specific model. The classification accuracy for each model is compared.

The first cluster model is related to motor imagery (MI) classification accuracy using a clustering-based model in the first band.

The second cluster model is related to motor imagery (MI) classification accuracy using a clustering-based model in the second band.

The third cluster model is related to motor imagery (MI) classification accuracy using a clustering-based model in the third band.

The first cluster model is marked as high performance, and medium accuracy is 68.8%.

That is, the first cluster model has a classification accuracy of 66.7% to 73.6%.

The first cluster model shows significantly high accuracy compared to the general model.

That is, the median accuracy of the general model is 64.6%, which is less than the median accuracy of 68.8% of the first cluster model.

In addition, in the case of the first cluster model, classification accuracy is significantly increased compared to the general model learned using all subjects without performing clustering.

In addition, there is no significant difference in performance between the first cluster model and the subject-specific model.

That is, the medium accuracy of the subject-specific model is 69.3%, showing a difference of about 0.5%, but not a significant difference in performance.

That is, when users are clustered using a specific band and the intentions of users in a group are classified, there is no difference in performance compared to when using an individual customized model.

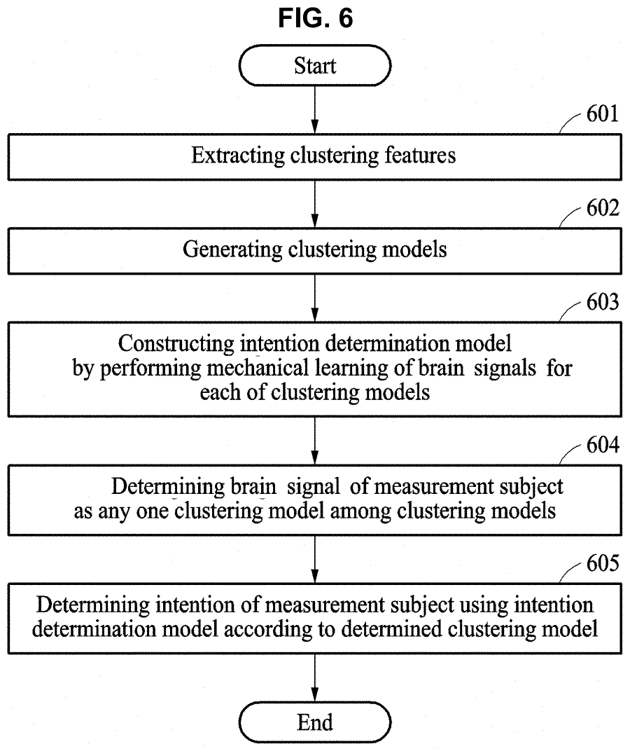

FIG. 6 is a flowchart for explaining a method of operating a brain-computer interface apparatus according to one embodiment of the present disclosure.

FIG. 6 illustrates a method of operating a brain-computer interface apparatus capable of minimizing a signal correction process between users using brain activity-based clustering technology according to one embodiment of the present disclosure.

Referring to FIG. 6, according to the method of operating a brain-computer interface apparatus according to one embodiment of the present disclosure, in step 601, a plurality of clustering features is extracted.

That is, according to the method of operating a brain-computer interface apparatus, a plurality of clustering features is extracted using frequency powers for each band of brain signals measured from a plurality of learning subjects.

According to the method of operating a brain-computer interface apparatus according to one embodiment of the present disclosure, in step 602, a plurality of clustering models is generated.

That is, according to the method of operating a brain-computer interface apparatus, a plurality of clustering models is generated based on a plurality of clustering features extracted in step 601.

According to the method of operating a brain-computer interface apparatus according to one embodiment of the present disclosure, in step 603, mechanical learning of brain signals for each of a plurality of clustering models is performed to construct an intention determination model.

That is, according to the method of operating a brain-computer interface apparatus, clustering of brain signals is performed according to a plurality of clustering models generated in step 602, and mechanical learning of brain signals is performed according to the clustering models to construct an intention determination model according to the clustering models.

According to the method of operating a brain-computer interface apparatus according to one embodiment of the present disclosure, in step 604, a brain signal of a measurement subject is determined as any one clustering model among a plurality of clustering models.

That is, according to the method of operating a brain-computer interface apparatus, brain signals are acquired when a measurement subject is in a resting state, and clustering is performed according to the clustering features of the acquired brain signals to classify clustering models.

According to the method of operating a brain-computer interface apparatus according to one embodiment of the present disclosure, in step 605, the intention of a measurement subject is determined using an intention determination model according to a clustering model.

That is, according to the method of operating a brain-computer interface apparatus, by using an intention determination model learned in advance for a clustering model, a process of measuring brain signals several times is omitted, and the intention of a measurement subject is determined.

Here, omitting the process of measuring brain signals several times may be related to minimization of a signal correction process.

Accordingly, the present disclosure may provide a brain-computer interface apparatus capable of minimizing, without deterioration in performance, a signal correction process by using clustering models designed using the brain signals of a user rather than individual customized models that take a lot of time and a method of operating the brain-computer interface apparatus.

The present disclosure can provide a brain-computer interface apparatus capable of minimizing a signal correction process between users using brain activity-based clustering technology and a method of operating the same.

The present disclosure can provide a brain-computer interface apparatus capable of minimizing, without deterioration in performance, a signal correction process by using clustering models designed using the brain signals of a user rather than individual customized models that take a lot of time and a method of operating the brain-computer interface apparatus.

The present disclosure can provide a brain-computer interface apparatus having high recognition performance for user intentions while minimizing a signal correction process by using customized clustering models and a method of operating the brain-computer interface apparatus.

The present disclosure can reduce time spent on classifying user intentions by extracting clustering features for designing clustering models using the frequency powers of brain signals measured from a plurality of learning subjects or a measurement subject.

The apparatus described above may be implemented as a hardware component, a software component, and/or a combination of hardware components and software components. For example, the apparatus and components described in the embodiments may be achieved using one or more general purpose or special purpose computers, such as, for example, a processor, a controller, an arithmetic logic unit (ALU), a digital signal processor, a microcomputer, a field programmable gate array (FPGA), a programmable logic unit (PLU), a microprocessor, or any other device capable of executing and responding to instructions. The processing device may execute an operating system (OS) and one or more software applications executing on the operating system. In addition, the processing device may access, store, manipulate, process, and generate data in response to execution of the software. For ease of understanding, the processing apparatus may be described as being used singly, but those skilled in the art will recognize that the processing apparatus may include a plurality of processing elements and/or a plurality of types of processing elements. For example, the processing apparatus may include a plurality of processors or one processor and one controller. Other processing configurations, such as a parallel processor, are also possible.

The software may include computer programs, code, instructions, or a combination of one or more of the foregoing, configure the processing apparatus to operate as desired, or command the processing apparatus, either independently or collectively. In order to be interpreted by a processing device or to provide instructions or data to a processing device, the software and/or data may be embodied permanently or temporarily in any type of a machine, a component, a physical device, a virtual device, a computer storage medium or device, or a transmission signal wave. The software may be distributed over a networked computer system and stored or executed in a distributed manner. The software and data may be stored in one or more computer-readable recording media.

The methods according to the embodiments of the present disclosure may be implemented in the form of a program command that can be executed through various computer means and recorded in a computer-readable medium. The computer-readable medium can store program commands, data files, data structures or combinations thereof. The program commands recorded in the medium may be specially designed and configured for the present disclosure or be known to those skilled in the field of computer software. Examples of a computer-readable recording medium include magnetic media such as hard disks, floppy disks and magnetic tapes, optical media such as CD-ROMs and DVDs, magneto-optical media such as floptical disks, or hardware devices such as ROMs, RAMs and flash memories, which are specially configured to store and execute program commands Examples of the program commands include machine language code created by a compiler and high-level language code executable by a computer using an interpreter and the like. The hardware devices described above may be configured to operate as one or more software modules to perform the operations of the embodiments, and vice versa.

Although the present disclosure has been described with reference to limited embodiments and drawings, it should be understood by those skilled in the art that various changes and modifications may be made therein. For example, the described techniques may be performed in a different order than the described methods, and/or components of the described systems, structures, devices, circuits, etc., may be combined in a manner that is different from the described method, or appropriate results may be achieved even if replaced by other components or equivalents.

Therefore, other embodiments, other examples, and equivalents to the claims are within the scope of the following claims.

### DESCRIPTION OF SYMBOLS

100: BRAIN-COMPUTER INTERFACE APPARATUS
110: BRAIN WAVE MEASUREMENT DEVICE
120: FEATURE EXTRACTOR
130: CLUSTERING MODEL GENERATOR
140: BRAIN WAVE PROCESSOR

What is claimed is:

1. A brain-computer interface apparatus capable of minimizing a signal correction process between users using clustering technology based on brain activity, the brain-computer interface apparatus comprising:
a feature extractor for extracting a plurality of clustering features using frequency powers for each band of brain signals measured from a plurality of learning subjects;
a clustering model generator for generating a plurality of clustering models based on the extracted clustering features; and
a brain wave processor for constructing an intention determination model by performing machine learning of brain signals for each of the generated clustering models, determining a newly measured brain signal of a measurement subject as any one of the clustering models, and determining an intention of the measurement subject using the constructed intention determination model.

2. The brain-computer interface apparatus according to claim 1, further comprising a brain wave measurement device for, using a plurality of measurement electrodes attached to a plurality of regions of the learning subjects or the measurement subject, measuring a plurality of brain signals for each of the regions,
wherein the brain signals exhibit different frequency powers according to the regions.

3. The brain-computer interface apparatus according to claim 2, wherein the brain wave measurement device measures brain signals having frequency powers of a first band at which a frequency power of 8 Hz to 12 Hz is measured, a second band at which a frequency power of 12 Hz to 18 Hz is measured, and a third band at which a frequency power of 18 Hz to 30 Hz is measured according to positions of the regions.

4. The brain-computer interface apparatus according to claim 3, wherein the feature extractor extracts at least one clustering feature of first clustering features corresponding to the first band, second clustering features corresponding to the second band, and third clustering features corresponding to the third band.

5. The brain-computer interface apparatus according to claim 1, wherein the feature extractor determines a plurality of clustering features for each band by averaging power spectral density (PSD) of the frequency powers for each band, and determines two clustering features among the determined clustering features through principal component analysis (PCA).

6. The brain-computer interface apparatus according to claim 5, wherein the clustering model generator generates the clustering models using the determined two clustering features and cluster analysis (k-means clustering).

7. The brain-computer interface apparatus according to claim 1, wherein the brain wave processor determines an intention of the measurement subject using an intention determination model corresponding to the determined clustering model, and thus reduces the signal correction process by reducing a machine learning process for the newly measured brain signal of the measurement subject.

8. The brain-computer interface apparatus according to claim 1, wherein the clustering model generator generates the clustering models so that inter-subject variability (ISV) of each of the clustering models is determined to be smaller than inter-subject variability (ISV) of all of the learning subjects.

9. A method of operating a brain-computer interface apparatus capable of minimizing a signal correction process between users clustering technology based on using brain activity, the method comprising:
extracting, in a feature extractor, a plurality of clustering features using frequency powers for each band of brain signals measured from a subjects;
generating, in a clustering model generator, a plurality of clustering models based on the extracted clustering features; and
constructing an intention determination model by performing machine learning of brain signals for each of the generated clustering models, determining a newly measured brain signal of a measurement subject as any one of the clustering models, and determining an intention of the measurement subject using the constructed intention determination model, in a brain wave processor.

10. The method according to claim 9, further comprising measuring, in a brain wave measurement device, a plurality of brain signals for each of a plurality of regions of the learning subjects or the measurement subject by using a plurality of measurement electrodes attached to the regions,
wherein the brain signals exhibit different frequency powers according to the regions.

11. The method according to claim 10, wherein the measuring of a plurality of brain signals for each of the regions comprises measuring brain signals having frequency powers of a first band at which a frequency power of 8 Hz to 12 Hz is measured, a second band at which a frequency power of 12 Hz to 18 Hz is measured, and a third band at which a frequency power of 18 Hz to 30 Hz is measured according to positions of the regions.

12. The method according to claim 9, wherein the determining of an intention of the measurement subject using the constructed intention determination model comprises reducing the signal correction process by reducing a machine learning process for the newly measured brain signal of the measurement subject as an intention of the measurement subject is determined using an intention determination model corresponding to the determined clustering model.
